(19) United States
(12) Patent Application Publication
(10) Pub. No.: US 2011/0313371 A1
(43) Pub. Date: Dec. 22, 2011

(54) MEDICATED, TRANSDERMAL FOOT PATCH

(76) Inventors: Aida Viana, Miami, FL (US); Lidia Viana, Miami, FL (US)

(21) Appl. No.: 13/112,892

(22) Filed: May 20, 2011

Related U.S. Application Data
(60) Provisional application No. 61/346,869, filed on May 20, 2010.

(51) Int. Cl. A61M 35/00

(52) U.S. Cl. 604/290; 604/307

(57) ABSTRACT

A medicated, transdermal foot patch includes a cushioning layer, and an adhesive layer comprised of an adhesive material that adheres the patch to the skin of the foot when the patch is applied to the foot with the adhesive layer in direct contact with the skin. A removable liner is placed over the adhesive layer to protect the adhesive layer until the adhesive layer is ready to be applied directly to the skin. One or more pain-relieving compounds is deliverable to the skin when the patch is applied to the foot with the adhesive layer is in direct contact with the skin.

### CROSS REFERENCE TO RELATED APPLICATION

This application claims the benefit of provisional application Ser. No. 61/346,869, filed May 20, 2010, the disclosure of which is incorporated herein by reference.

### TECHNICAL FIELD

The invention relates to a disposable, self-adhesive, medicated, transdermal foot patch for relieving and preventing foot pain caused by wearing high heels or uncomfortable shoes, which adheres directly to a bare foot.

### BACKGROUND

A transdermal patch, or skin patch, is a medicated adhesive patch that is placed on the skin to deliver a specific dose of medication through the skin and into the bloodstream. Often, this promotes healing to an injured area of the body. An advantage of a transdermal drug delivery route over other types such as oral, topical, etc. is that it provides a controlled release of the medicament into the patient. A disadvantage to development, however, stems from the fact that the skin is a very effective barrier. A wide variety of pharmaceuticals can be delivered by transdermal patches.

The main components to a transdermal patch are: liner, drug, adhesive, membrane, and backing. The liner protects the patch during storage and is removed prior to use. The drug is typically a drug solution in direct contact with a release liner. The adhesive serves to adhere the components of the patch together, along with adhering the patch to the skin. The membrane controls the release of the drug from the reservoir and multi-layer patches. The backing protects the patch from the outer environment.

There are five main types of transdermal patches, and these are: single-layer drug-in-adhesive; multi-layer drug-in-adhesive; reservoir; matrix; and vapor patch. The adhesive layer of a single-layer drug-in-adhesive system also contains the drug. In this type of patch the adhesive layer not only serves to adhere the various layers together, along with the entire system to the skin, but is also responsible for the releasing of the drug. The adhesive layer is surrounded by a temporary liner and a backing.

The multi-layer drug-in adhesive patch is similar to the single-layer system in that both adhesive layers are also responsible for the releasing of the drug. One of the layers is for immediate release of the drug and other layer is for controlled release of drug from the reservoir. The multi-layer system is different, however, in that it adds another layer of drug-in-adhesive, usually separated by a membrane (but not in all cases). This patch also has a temporary liner-layer and a permanent backing.

Unlike the single-layer and multi-layer drug-in-adhesive systems, the reservoir transdermal system has a separate drug layer. The drug layer is a liquid compartment containing a drug solution or suspension separated by the adhesive layer. This patch is also backed by the backing layer. In this type of system, the rate of release is zero order.

The matrix patch is also known as a monolithic device. The matrix system has a drug layer of a semisolid matrix containing a drug solution or suspension. The adhesive layer in this patch surrounds the drug layer partially overlaying it.

In a vapor patch, the adhesive layer not only serves to adhere the various layers together, but also to release vapor. The vapor patches release essential oils for up to six hours, and they are used primarily for decongestion. Other examples of vapor patches are controller vapor patches that improve the quality of sleep, and vapor patches that reducing cigarette smoking.

Transdermal patches have not been useful for application to the palms of the hands or soles of the feet due in part to increased thickness of the skin in these regions, which make absorption of medication more difficult. Another reason that transdermal patches have not been useful for application to these regions is due to the frequent flexing of the hands and feet in the course of a normal day, which makes it difficult for the patch to remain adhered to the skin of the sole of the foot or the palm of the hand with a transdermal patch adhesive suitable for dispersion of medication. Thus, users of transdermal patches have typically been directed to apply the patches in other regions of the body. One notable exception is a detox foot patch that is applied to the soles of the feet overnight, when the feet are not being used for walking or running.

### SUMMARY

A medicated, transdermal foot patch includes an adhesive layer comprised of an adhesive material that adheres the patch to the skin of the foot when the patch is applied to the foot with the adhesive layer in direct contact with the skin. A removable liner is placed over the adhesive layer to protect the adhesive layer until the adhesive layer is ready to be applied directly to the skin. One or more pain-relieving compounds is deliverable to the skin when the patch is applied to the foot with the adhesive layer is in direct contact with the skin.

In some embodiments, a cushioning layer or layers is provided. This cushioning layer can be combined with the foot patch in one or more ways. One way in which the cushioning layer can be combined with the patch is by use of the cushioning layer (e.g., moleskin, memory foam, etc.) as a backing of the patch, thus providing the foot patch with foot pad functionality. The cushioning layer can be of larger or different dimensions than those of the patch to allow the cushioning layer to also adhere to the skin. In some embodiments, a different, stronger adhesive can be employed to adhere the cushioning layer to the skin, thus assisting in holding the patch in place. Another way in which the cushioning layer can be combined with the patch is by application of the cushioning layer as a separate or integral foot pad atop the patch. In some embodiments, the cushioning layer can be adhered to the backing of the patch, and it can be of larger diameter and/or different shape than the patch so that it can additionally adhere directly to the skin of the foot, thereby assisting in holding the patch in place. In other embodiments, the separate foot pad can be employed inside a shoe without adhesion to the patch, which can still assist the patch in remaining in place by preventing sliding of the foot within the shoe.

### BRIEF DESCRIPTION OF THE DRAWINGS

FIG. 1 is a plan view of the representative example of a disposable, self-adhesive, medicated, transdermal foot patch having an integral cushioning layer employed as a backing of the patch.

FIG. 2 is a cross section of the representative example of 1 disposable, self-adhesive, medicated, transdermal foot patch of FIG. 1.

FIG. 3 is a perspective view of a representative sample of a disposable, self-adhesive, medicated, transdermal foot patch for use with a separate foot pad.

### DETAILED DESCRIPTION OF EXEMPLARY EMBODIMENTS

In the following description, like numbers refer to like elements.

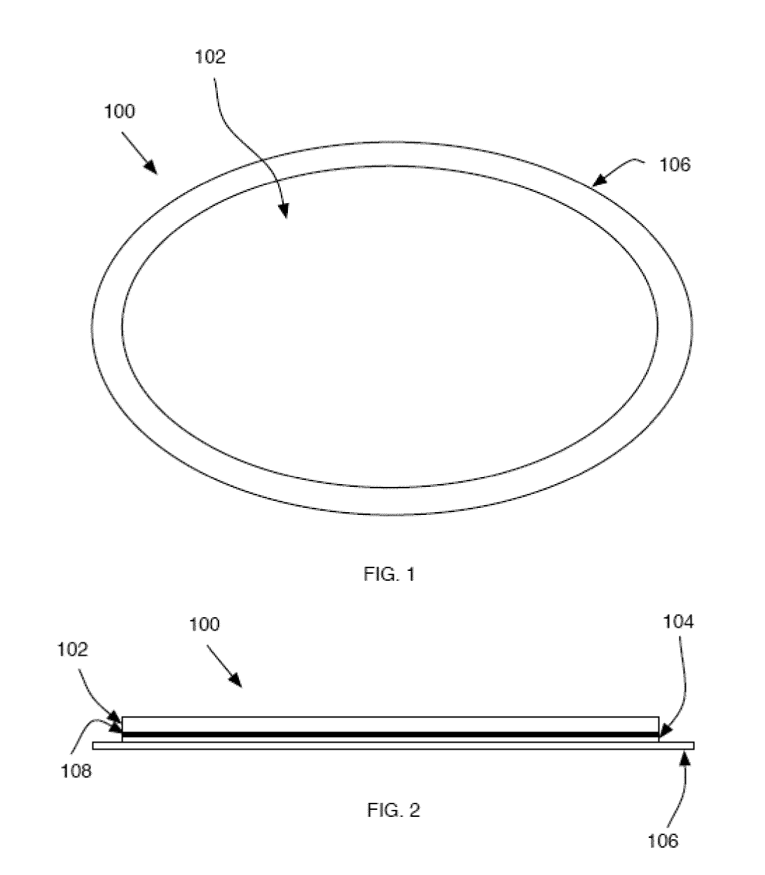

FIGS. 1-2 illustrate some examples of a disposable, self-adhesive, medicated, transdermal foot patch 100. The patch comprises at least a cushioning layer 102 and an adhesive layer 104. The layers shown in FIG. 2 are not to scale. The adhesive layer is comprised of an adhesive material that adheres the patch to the skin of the foot when the patch is applied to the foot. The adhesive layer is in direct contact with the skin. A liner 106 is placed over the adhesive layer to protect until the adhesive layer is ready to be applied directly to the skin, at which time the liner is removed.

The adhesive layer 104 is impregnated with one or more pain-relieving compounds. In one example, at least one of the pain-relieving compounds is comprised of at least one of capsaicin, glucosamine, methyl salicylate, menthol, camphor, and tea tree oil. The adhesive layer is, in one embodiment, designed to release the dose of the one or more pain-relieving compounds immediately or, in an alternate embodiment, in a controlled manner over a predetermined period of time when the patch 100 is placed on the skin of a foot, such as the sole of the foot. The pain-relieving compounds are capable of at least partially being absorbed by the foot through the skin. The adhesive layer may be impregnated with additional active and/or inactive compounds, for example, antifungal agents. Depending on the function or purpose of an additional compound, it may or may not be of a type that is absorbed by the skin.

The cushioning layer 102 functions to protect the foot by reducing the effects of pressure and/or friction from the shoe on the foot. In one embodiment, the cushioning layer 102 is made of one or more layers of moleskin. In another embodiment, the cushioning layer is made of one or more layers of memory foam. Alternately, the cushioning layer is made of multiple layers of different materials. In the example given above, the cushioning layer is made relatively thin to avoid making a shoe feel tight. The patch is, in one embodiment, sized and shaped so that it can be discretely worn with both open and closed style shoes. The patch 100 is, in another embodiment, sized and shaped to fit a particular area of the foot, for example a ball, heel, or a side of the foot. The shape of the patch 100 shown in FIG. 1 is representative generally of a patch and is not intended to represent a shape for any particular area of the foot.

The exterior top surface of the patch, which in the example is the top surface of cushioning layer 102, but could also be another layer of a different material or a surface treatment of the cushioning layer, has, optionally, a greater coefficient of friction than a bare foot and reduces the tendency of the foot to slide forward within the shoe.

In the illustrated embodiment, an additional layer or membrane 108 is placed between the adhesive layer and the cushioning layer for preventing or substantially reducing migration into the cushioning layer active compounds impregnating the adhesive.

In an alternative embodiment that is not shown, the patch may be formed with a reservoir containing one or more compounds for transdermal delivery. The reservoir is, for example, formed between the cushioning layer and the adhesive layer, in which one or more compounds are contained for controlled release through a membrane to which an adhesive layer is applied. The adhesive layer may or may not include compounds for transdermal delivery. The reservoir and/or the adhesive layer contains at least one pain-relieving compound selected from the group consisting of capsaicin, glucosamine, methyl salicylate, menthol, camphor and tea tree oil, which compound will be delivered to the skin when the patch is applied to the foot. The reservoir and/or the adhesive layer may contain additional active and/or inactive compounds, and each of the adhesive layer and reservoir may contain a different pain-relieving compound.

In another alternative embodiment, the at least one pain-relieving compound is contained in solution or suspension in a semisolid matrix that provides for controlled release of the compound. The adhesive layer surrounds the drug layer and may partially overlay it. The at least one pain-relieving compound selected from the group consisting of capsaicin, glucosamine, methyl salicylate, menthol, camphor and tea tree oil.

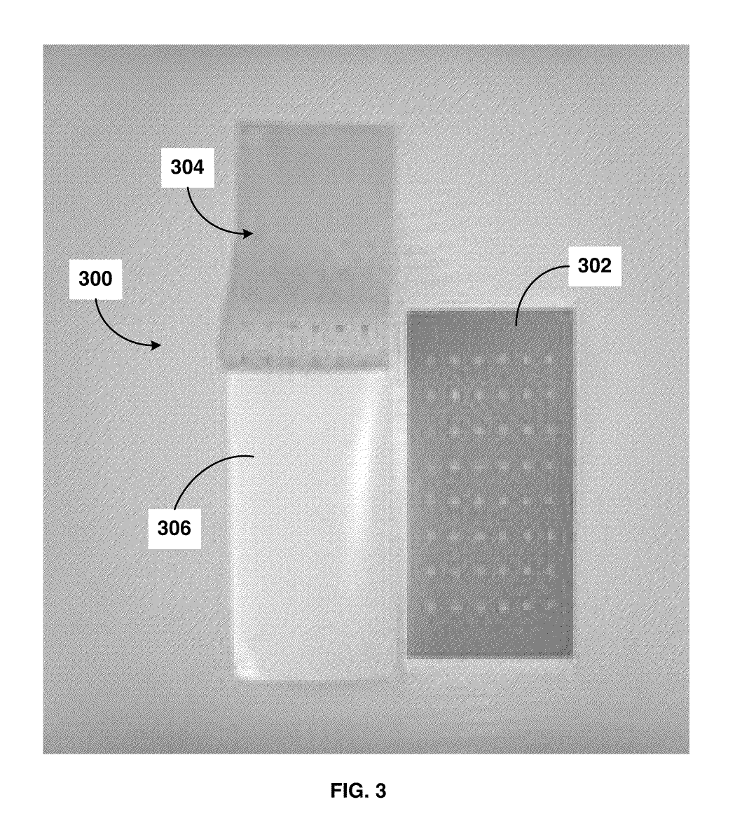

Turning now to FIG. 3, in other embodiments, a medicated, transdermal foot patch 300 can be provided that is an analgesic and a mild anti-inflammatory product. Once applied, its ingredients are absorbed into the skin to stimulate blood circulation, which helps to expedite the healing process. Its quickly permeating, and warm action provides speedy relief and comfort for hours.

In some embodiments, the patch 300 has a stretchable backing 302 and an adhesive layer 304. The layers shown in FIG. 3 are not to scale. The adhesive layer is comprised of an adhesive material that adheres the patch to the skin of the foot when the patch is applied to the foot. The adhesive layer is in direct contact with the skin. A liner 306 is placed over the adhesive layer to protect until the adhesive layer is ready to be applied directly to the skin, at which time the liner is removed.

The adhesive layer 304 is impregnated with one or more pain-relieving compounds. In one example, at least one of the pain-relieving compounds is comprised of at least one of capsaicin, glucosamine, methyl salicylate, menthol, camphor, and tea tree oil. The adhesive layer is, in one embodiment, designed to release the dose of the one or more pain-relieving compounds immediately or, in an alternate embodiment, in a controlled manner over a predetermined period of time when the patch 300 is placed on the skin of a foot, such as the sole of the foot. The pain-relieving compounds are capable of at least partially being absorbed by the foot through the skin. The adhesive layer may be impregnated with additional active and/or inactive compounds, for example, antifungal agents. Depending on the function or purpose of an additional compound, it may or may not be of a type that is absorbed by the skin.

The patch is, in one embodiment, sized and shaped so that it can be discretely worn with both open and closed style shoes. The patch 300 is, in another embodiment, sized and shaped to fit a particular area of the foot, for example a ball, heel, or a side of the foot. The shape of the patch 300 shown in FIG. 3 is representative generally of a patch and is not intended to represent a shape for any particular area of the foot.

In an alternative embodiment that is not shown, the patch may be formed with a reservoir containing one or more compounds for transdermal delivery. The reservoir is, for example, formed between the cushioning layer and the adhesive layer, in which one or more compounds are contained for controlled release through a membrane to which an adhesive layer is applied. The adhesive layer may or may not include compounds for transdermal delivery. The reservoir and/or the adhesive layer contains at least one pain-relieving compound selected from the group consisting of capsaicin, glucosamine, methyl salicylate, menthol, camphor and tea tree oil, which compound will be delivered to the skin when the patch is applied to the foot. The reservoir and/or the adhesive layer may contain additional active and/or inactive compounds, and each of the adhesive layer and reservoir may contain a different pain-relieving compound.

In another alternative embodiment, the at least one pain-relieving compound is contained in solution or suspension in a semisolid matrix that provides for controlled release of the compound. The adhesive layer surrounds the drug layer and may partially overlay it. The at least one pain-relieving compound selected from the group consisting of capsaicin, glucosamine, methyl salicylate, menthol, camphor and tea tree oil.

The cushioning layer (not shown) can function to protect the foot by reducing the effects of pressure and/or friction from the shoe on the foot. In one embodiment, the cushioning layer is a foot pad that is sized and shaped for use with the patch 300. In one embodiment, the cushioning layer can be made of one or more layers of moleskin. In another embodiment, the cushioning layer is made of one or more layers of memory foam. Alternately, the cushioning layer is made of multiple layers of different materials. In the example given above, the cushioning layer is made relatively thin to avoid making a shoe feel tight. The combination of the cushioning layer and patch 300 is, in one embodiment, sized and shaped so that it can be discretely worn with both open and closed style shoes. The combination of the cushioning layer and the patch 300 is, in another embodiment, sized and shaped to fit a particular area of the foot, for example a ball, heel, or a side of the foot.

In some embodiments, the patch 300 and its foot pad can be packaged together as a kit. For example, a foot pad of larger and/or different dimensions can be applied to the backing of the patch 300 prior to sale, and a single liner can be provided to the pad and patch 300 thus combined. Alternatively or additionally, the pad and patch can have individual liners, and packaged together with instructions to purchasers to apply the foot pad atop the patch 300 either before or after application of the patch 300 to the skin of the foot, such as the sole of the foot. Alternatively, or additionally, purchasers can be instructed to cut the patch and/or pad to desired dimensions for application as above, and the instructions can admonish the purchasers to ensure that the pad is of dimensions larger and/or different from those of the patch 300, so as to permit application of the pad atop the patch 300 while permitting both the pad and the patch to adhere directly to the skin. In additional or alternative embodiments, the pad and patch can be packaged and sold separately, but with clear instructions to purchasers to purchase both the pad and the patch 300 and to employ the pad and patch 300 together. For example, the products can be sold in close proximity to one another, such as on the same or a nearby shelf, or on a common end cap or display, with the instructions on one of the packages of one of the products. Alternatively or additionally, the products can be sold together via a web storefront providing a display promoting the products for use together. In yet additional or alternative embodiments, the purchaser can be instructed to employ the separate pad inside a shoe without adhesion to the patch, which can still assist the patch in remaining in place by preventing sliding of the foot within the shoe.

The foregoing description is of exemplary and preferred embodiments employing teachings of the invention. However, the invention is not limited to the described embodiments. Alterations and modifications to the disclosed embodiments may be made without departing from the invention. The meaning of the terms used in this specification are, unless expressly stated otherwise, intended to have ordinary and customary meaning and are not intended to be limited to the details of the illustrated structures or the disclosed embodiments.

What is claimed is:

1. A medicated, transdermal foot patch, comprising:
an adhesive layer comprised of an adhesive material that adheres the patch to the skin of the foot when the patch is applied to the foot with said adhesive layer in direct contact with the skin;
a removable liner placed over said adhesive layer to protect said adhesive layer until said adhesive layer is ready to be applied directly to the skin; and
one or more pain-relieving compounds deliverable to the skin when the patch is applied to the foot with said adhesive layer is in direct contact with the skin.

2. The medicated, transdermal foot patch of claim 1, wherein said adhesive layer is impregnated with said one or more pain-relieving compounds.

3. The medicated, transdermal foot patch of claim 2, wherein said adhesive layer is designed to release a dose of said one or more pain-relieving compounds immediately upon application of said adhesive layer directly to the skin of the foot.

4. The medicated, transdermal foot patch of claim 2, wherein said adhesive layer is designed to release a dose of said one or more pain-relieving compounds in a controlled manner over a predetermined period of time while said adhesive layer is applied directly to the skin of the foot.

5. The medicated, transdermal foot patch of claim 2, further comprising:
a membrane layer operatively positioned between said adhesive layer and a cushioning layer employed as a backing of said patch to at least one of prevent or substantially reduce migration into said cushioning layer of active compounds impregnating said adhesive layer.

6. The medicated, transdermal foot patch of claim 1, wherein said adhesive layer is impregnated with at least one antifungal agent.

7. The medicated, transdermal foot patch of claim 1, wherein the patch is formed with a reservoir containing said one or more pain-relieving compounds for transdermal delivery.

8. The medicated, transdermal foot patch of claim 7, wherein the reservoir is formed between a cushioning layer employed as a backing of said patch and said adhesive layer, in which said one or more pain-relieving compounds are contained for controlled release through a membrane to which said adhesive layer is applied.

9. The medicated, transdermal foot patch of claim 1, wherein said at least one pain-relieving compound is contained in at least one of solution or suspension in a semisolid matrix that provides for controlled release of said at least one pain-relieving compound.

10. The medicated, transdermal foot patch of claim 1, wherein at least one of said one or more pain-relieving compounds is comprised of at least one of capsaicin, glucosamine, methyl salicylate, menthol, camphor, or tea tree oil.

11. The medicated, transdermal foot patch of claim 1, further comprising:
a cushioning layer.

12. The medicated, transdermal foot patch of claim 1, wherein said cushioning layer is employed as a backing of said patch.

13. The medicated, transdermal foot patch of claim 11, wherein said cushioning layer is made of one or more layers of moleskin.

14. The medicated, transdermal foot patch of claim 11, wherein said cushioning layer is made of one or more layers of memory foam.

15. The medicated, transdermal foot patch of claim 11, wherein an exterior top surface of said cushioning layer has a greater coefficient of friction than a bare foot, thereby reducing tendency of the foot to slide forward within a shoe.

16. A method of using a medicated, transdermal foot patch, the method comprising:
removing a removable liner placed over an adhesive layer of the patch to protect the adhesive layer until the adhesive layer is ready to be applied directly to skin; and
adhering the patch to skin of a foot by applying the patch to the foot with the adhesive layer of the patch in direct contact with the skin, thereby delivering one or more pain-relieving compounds to the skin when the patch is applied to the foot with the adhesive layer is in direct contact with the skin.

17. The method of claim 16, wherein the adhesive layer is impregnated with said one or more pain-relieving compounds.

18. The method of claim 17, wherein the adhesive layer is designed to release a dose of the one or more pain-relieving compounds immediately upon application of the adhesive layer directly to the skin of the foot.

19. The method of claim 17, wherein the adhesive layer is designed to release a dose of said one or more pain-relieving compounds in a controlled manner over a predetermined period of time while the adhesive layer is applied directly to the skin of the foot.

20. The method of claim 16, wherein adhering the patch to skin of a foot further includes introducing an antifungal agent impregnating the adhesive layer to the skin of the foot.

21. The method of claim 16, wherein delivering the one or more pain-relieving compounds to the skin includes delivering at least one of capsaicin, glucosamine, methyl salicylate, menthol, camphor, or tea tree oil.

22. The method of claim 16, further comprising:
following instructions included with the patch by combining the patch with a cushioning layer.

23. The method of claim 22, wherein the cushioning layer is made of one or more layers of moleskin.

24. The method of claim 22, wherein the cushioning layer is made of one or more layers of memory foam.

25. The method of claim 22, wherein an exterior top surface of the cushioning layer has a greater coefficient of friction than a bare foot, thereby reducing tendency of the foot to slide forward within a shoe.

26. A medicated, transdermal foot patching kit, comprising:
a medicated, transdermal foot patch;
a cushioning layer; and
instructions to a purchaser of the kit to employ the cushioning layer with the medicated, transdermal patch.

27. The kit of claim 26, wherein said cushioning layer has dimensions different from dimensions of said medicated, transdermal foot patch, such that an adhesive of said cushioning layer adheres directly to skin of a foot when applied atop said medicated, transdermal foot patch to the foot.

28. The kit of claim 26, wherein said cushioning layer is made of one or more layers of moleskin.

29. The kit of claim 26, wherein said cushioning layer is made of one or more layers of memory foam.

30. The kit of claim 26, wherein an exterior top surface of the cushioning layer has a greater coefficient of friction than a bare foot, thereby reducing tendency of the foot to slide forward within a shoe.
